(19) United States
(12) Patent Application Publication
(10) Pub. No.: US 2006/0159640 A1
(43) Pub. Date: Jul. 20, 2006

(54) Antiperspirant gripping composition

(76) Inventor: Larry Plunk SR., Port Arthur, TX (US)

Correspondence Address: PATEL & ALUMIT, P.C., SUITE 360, 16830 VENTURA BOULEVARD, WOODLAND HILL, CA, 91364, US

(21) Appl. No.: 11/298,131

(22) Filed: Dec. 8, 2005

Related U.S. Application Data
(60) Provisional application No. 60/635,222, filed on Dec. 9, 2004.

(51) Int. Cl. A61K 8/26

(52) U.S. Cl. 424/68

(57) ABSTRACT

An antiperspirant gripping compound with astringent properties designed to be applied topically to the skin of the hands to enhance the grip of a user on tools, sports equipment or other surfaces. The antiperspirant gripping compound consists of approximately 23% aluminum chlorohydroxide dihydrate, and aluminum hydroxyl chloride dihydrate solution mixed with a base, for example water, or a specially formulated base.

### CROSS-REFERENCE TO RELATED APPPLICATION

The present application claims priority to U.S. Provisional Patent Application No. 60/635,222, filed Dec. 9, 2004, which is hereby incorporated by reference as if set forth in full herein.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The present invention relates to antiperspirant compounds, and more particularly to a topical solution that is applied to a person's hands to keep them dry and improve the grip on articles, particularly sports equipment.

Antiperspirant compositions are well known in the art known in the art and are used in various industries. Antiperspirants in the cosmetic industry and chemical literature are usually formulated for application to various parts of the body in the form of creams, gels, solids, roll-ons and powders. These compositions are typically not suitable for application to the hands. For example, some compositions may be tacky and produce undesirable levels of buildup material on the skin. Other compositions may be difficult to apply, such as those in powder forms; while other compositions are not suitable due to irritation to sensitive areas of the skin.

A person's hands are subject to similar perspiration problems exhibited by other parts of the body. Additionally, the skin of the hand can often dry out and crack, requiring a mild treating agent and skin conditioner in addition to antiperspirant. As controlling the perspiration of the hands is critical in many occupations and activities; such as driving vehicles, performing delicate operations, and in most sporting activities, a composition is needed that can treat perspiration. Moreover, the gripping portion of tools and sporting equipment is generally made of materials that may become difficult to grasp when wet, particularly due to perspiration.

In an attempt to improve the grip on various surfaces, materials that can be applied to the hand or the implement to be gripped have been developed. For example, the rosin bag is a traditional piece of equipment used in baseball by both pitchers and hitters to improve their grip on baseballs and bats. Similarly, some tennis and golf players lightly apply powdered rosin to their hands to improve the grip on a tennis racket. In other sports, athletes rub magnesia or chalk to their hands to dry them out.

The fundamental disadvantages of the compositions known in the art however, are their lack of staying power, permanence and relatively poor effectiveness. Perspiration on the bare hands of an athlete, for example, can easily dislodge the rosin dust, which subsequently requires the frequent reapplication of rosin. To avoid these drawbacks, the several materials have been described that purportedly improve gripping characteristics.

U.S. Pat. No. 5,468,473 to Mullen, discloses an aqueous alcohol carrier solvent based antiperspirant composition which does not contain silicone products and which forms a thick lotion for use on the hands and feet. The composition includes a thickener-absorber mixture of hydroxylalkylcellulose, preferably hydroxypropylmethylcellulose, with colloidal silica and silicates and an antiperspirant active material, preferably chlorhydrol. A preferred embodiment includes the addition of Tea Tree Oil to the formulation. The antiperspirant composition reduces sweating on the hands and feet and is nonirritating, nontacky and mildly soothing to the skin. a nonirritating antiperspirant thick lotion for the hands and feet having maximum antiperspirant activity at low concentrations is described.

U.S. Pat. No. 6,656,257 to Cohen, describes a composition for improving grip between the hands and a tool or implement, which comprises a chlorinated paraffin, a dispersing agent, and a solvent in an amount up to and including about 3.0% by weight relative to the total weight of the composition. A method of making the composition in the form of a dry powder, as well as a method of using the dry powder to improve friction or grip on the hands, is also provided.

In U.K. Pat. No. 2,120,270 to Schneider, a hand composition for improving the holding of sports equipment is described and is particularly suited for use in tennis and other types of activities in which a racket is held in the hand.

Although these compositions serve a similar purpose, they are not of the type that the present invention embodies. Therefore, it is an object of the present invention to overcome the shortcomings of in the art and to provide a hand topical solution that has antiperspirant properties. It is a further object of the present invention to provide a hand topical antiperspirant that is easy to use, and effectively increases the gripping properties of the skin. These and other objects are set forth in the following detailed description and claims.

### SUMMARY

The present invention relates to a topical solution for use on a person's hands to keep them dry and for improving the holding of articles, particularly sporting equipment. The composition preferably comprises approximately 23% aluminum chlorohydroxide dihydrate and aluminum hydroxy chloride dihydrate solution mixed with a specially formulated base, for instance water.

Although particular embodiments of the present invention have been described in the foregoing description, it is to be understood that the present invention is not to be limited to just the embodiments disclosed, but that they are capable of numerous rearrangements, modifications and substitutions without departing from the description herein.

### FIGURES

None.

### REFERENCE NUMBERS

None.

### DETAILED DESCRIPTION

An improved gripping composition of the present invention is a topical solution to be used on a person's hands during sporting activities, such as golf, to keep them dry. The composition or the product of the present invention can be in various forms such as powder form or in the form of an aerosol and is not restricted to a solution form. Although the percentage may vary, the composition preferably comprises 23% aluminum chlorohydroxide dihydrate and aluminum hydroxy chloride dihydrate solution mixed with a specially formulated base, i.e., water. Additionally, the composition is not limited to use during sporting activities or only to the hands of a user.

Aluminum chlorohydroxide or aluminum hydroxy chloride [Al₂Cl(OH)₅] has astringent properties and hinders sweat secretion through the skin when applied topically. In one preferred composition, it is hydrolyzed in an alkaline medium to achieve a weakly low pH value. This is the preferred embodiment of the invention when water is used as the substrate medium. Due to its astringent action, the composition in accordance with the present invention prevents perspiration over a longer period of time than those of the prior art.

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

Although preferred embodiments of the present invention have been shown and described, various modifications and substitutions may be made thereto without departing from the spirit and scope of the invention. Accordingly, it is to be understood that the present invention has been described by way of illustration and not limitation.

What is claimed is:

1. An antiperspirant gripping compound comprising aluminum chlorohydroxide dihydrate and aluminum hydroxy chloride dihydrate solution mixed with a base substrate.

2. An antiperspirant gripping compound comprising between 20% and 30%, preferably 23% aluminum chlorohydroxide dihydrate and aluminum hydroxy chloride dihydrate solution mixed with a base substrate.

3. An antiperspirant gripping compound comprising 23% aluminum chlorohydroxide dihydrate and aluminum hydroxy chloride dihydrate solution mixed with a base substrate.

4. the antiperspirant gripping compound of claim 1, wherein the base substrate is water.

5. The antiperspirant gripping compound of claim 2, wherein the base substrate is water.

6. The antiperspirant gripping compound of claim 3, wherein the base substrate is water.

7. The antiperspirant gripping compound of claim 1, wherein the composition has a pH value lower than 6, to achieve mild alkaline conditions.

8. The antiperspirant gripping compound of claim 2, wherein the composition has a pH value lower than 6 to achieve mild alkaline conditions.

9. The antiperspirant gripping compound of claim 3, wherein the composition has a pH value lower than 6 to achieve mild alkaline conditions.
